(19) United States
(12) Patent Application Publication
(10) Pub. No.: US 2003/0050255 A1
(43) Pub. Date: Mar. 13, 2003

(54) Organic compounds

(76) Inventors: Michelle Pik-han Wong, Duluth, GA (US); Kasey Jon Minick, Morrisville, NC (US); Shau-fong Yen, Atlanta, GA (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/227,086

(22) Filed: Aug. 23, 2002

Related U.S. Application Data
(60) Provisional application No. 60/314,448, filed on Aug. 23, 2001.

(51) Int. Cl. A61K 31/70; A61K 9/14

(52) U.S. Cl. 514/33; 424/486; 424/487

(57) ABSTRACT

This invention is directed to ophthalmic compositions comprising an ascomycin for once-a-day administration.

This invention relates to ophthalmic compositions comprising anascomycin or a compound of the FK 506 class for once-a-day administration.

It has been found that ophthalmic compositions often have to be applied two to four times a day and that such repeated administration is not optimal in practice because for optimal treatment the patient has to have constant access to the composition and the patient is disturbed several times a day. This type of multiple administration of a drug, in particular of an ophthalmic composition, frequently leads to the problems of overdosing and underdosing. Overdosing may generate ocular irritation, whereas underdosing may lead to a reoccurrence of the symptoms.

There is thus a need for compositions that allow once-a-day administration of ophthalmic drugs.

FK506 is a known macrolide antibiotic that is produced by Streptomyces tsukubaensis No 9993. It is also a potent immunosuppressant. The structure of FK506 is given in the appendix to the Merck Index, 11th Edition as item A5. Methods of preparing FK506 are described in EP 184162. A large number of derivatives, antagonists, agonists and analogues of FK506, which retain the basic structure and at least one of the biological properties (for example immunological properties) of FK506, are known. These compounds are described in a large number of publications, for example EP 184162, EP 315978, EP 323042, EP 423714, EP 427680, EP 465426, EP 474126, WO 91/13889, WO 91/19495, EP 484936, EP 532088, EP 532089, EP 569337, EP 626385, WO 93/5059 and the like. Ascomycins and derivatives thereof, including FK506, are referred to hereinafter as “ascomycins”.

Preferred ascomycins for use in the present invention include FK506; 33-epi-chloro-33-desoxy-ascomycin as disclosed in Example 66a in EP 427680 (hereinafter referred to as Compound A); {[1E-(1R,3R,4R)]1R,4S,5R,6S,9R,10E,13S,15S,16R,17S,19S,20S}-9-ethyl-6,16, 20-trihydroxy-4-[2-(4-hydroxy-3-methoxy-cyclohexyl)-1-methylvinyl]-15,17-dimethoxy-5,11,13,19-tetramethyl-3-oxa-22-aza-tricyclo[18.6.1.0(1,22)]heptacos-10-ene-2,8,21,27-tetraone as disclosed in Examples 6d and 71 in EP 569 337; and {1R,5Z,9S,12S-[1E-(1R,3R,4R)],13R,14S,17R,18E,21S,23S,24R,25S,27R} 17-ethyl-1,14-dihydroxy-12-[2-(4-hydroxy-3-methoxy-cyclohexyl)-1-methylvinyl]-23,25-dimethoxy-13,19,21,27-tetramethyl-11,28-dioxa-4-aza-tricyclo[22.3.1.0(4,9)]octacosa-5,18-diene-2,3,10,16-tetraone, also known as 5,6-dehydro-ascomycin as disclosed in Example 8 in EP 626 385. Particularly preferred is Compound A.

Applicants have found that compositions comprising an ascomycin may be formulated for once-a-day administration which provide for a therapeutic effect of the ascomycin in the eye over 24 hours and that such compositions are surprisingly well tolerated. Moreover, administration of such once-a-day compositions produce a highly reliable and more reproducible clinical result in patients.

Therefore, in one aspect the present invention provides an ophthalmic composition comprising an ascomycin that is adapted for topical once-a-day administration (hereinafter compositions of the present invention).

The concentration of the ascomycin is preferably between about 0.005% and about 3.0%, more preferably between about 0.01% and about 1.5% by weight based on the total weight of the composition. If desired, the compositions of the present invention may be in the form of a suspension, which e.g., contain particles of an ascomycin with a mean particle diameter between about 0.2 and about 900 nm.

Applicants have found that compositions of the present invention with moderate viscosity, e.g. between about 50 and about 2000, or e.g. between about 100 and about 1000 mPa s at 20 to 25° C., are comfortable to apply to the eye. Upon instillation into the eye, the viscosity of the compositions of the present invention may increase or decrease depending on the formulation. Preferably the viscosity decreases upon instillation into the eye. The compositions of the present invention have excellent retention after instillation into the eye, i.e., they are retained in the tear film and do not drain into the nose.

The compositions of the present invention may comprise pharmaceutically acceptable excipients that are suitable for ophthalmic compositions. For example the excipients and/or compositions of the present invention should not significantly affect the lacrimal system nor the ocular tear film.

Information on the properties, specifications and characteristics of ophthalmically acceptable excipients are described, e.g., in standard texts such as Fiedler, H. P.; 1996; Lexikon der Hilfsstoffe für Pharmazie, Kosmetik und angrenzende Gebiete; Editio Cantor Verlag Aulendorf (Germany), and Kibbe, A. H.; 2000; Handbook of Pharmaceutical Excipients, a joint publication of Pharmaceutical Press, London (UK), and American Pharmaceutical Association, Washington (US) as well as manufacturers' brochures, the contents of which are incorporated herein by reference.

The compositions of the present invention may comprise (1) biocompatible polymers (thickeners). Such a polymer may be a thermo-reversible polymer, e.g., one which increases the viscosity of the composition on increasing temperature. Additionally such polymers may exhibit muco-adhesive properties.

A wide variety of polymers may be chosen but it has been found that the following are particularly useful.

1.1 polyoxyethylene-polyoxypropylene copolymers and preferably block co-polymers. Preferably the polyoxypropylene polymer number is from about 10 to about 60. Preferably the polyoxyethylene polymer number is from about 10 to about 150. Examples include such as those known and commercially available under the trade names Lutrol® and Poloxamer® (Fiedler, loc. cit., p. 1200, 1203; Handbook of Pharmaceutical Excipients, loc. cit., page 386) and in particular Poloxamer® 407 and Lutrol® F127, having a melting point of about 52 to 57° C.

1.2 acrylic acid homo- and co-polymers, which preferably are cross-linked; preferably carboxypolymethylene. Preferred molecular weights are between about 500,000, preferably from 1,000,000 to about 10,000,000 Daltons. Preferably, the acid groups comprise between 56 and 68% by weight of the total polymer.

Preferably it is crosslinked with a polyol, e.g.

1.2.1 with divinyl glycol, such as those known and commercially available under the trade name Noveon® from BFGoodrich, and in particular Noveon® AA-1, or

1.2.2 with allylsucrose or allypentaerythritol, such as those known and comercially available under the trade name Carbopol® from BFGoodrich. Examples include those known and commercially available under the trade name Carbopol® and in particular Carbopol® 934P,974P,980,981 and 984.

The exact amounts of polymer/thickener components may vary within wide limits to produce a composition of the present invention within the viscosity indicated above. The amount may be between about 0.05% and about 10% by weight of the total composition, e.g., the polyoxyethylene-polyoxypropylene copolymers can be present at a concentration of about 0.1%, and the acrylic acid homo- and co-polymers, preferably cross-linked, can be present at concentrations between 0.1% and 0.2%, such as 0.15%, such as a Carbopol at a concentration of 0.15%.

Preferably the present invention provides ophthalmic compositions comprising anascomycin and at least one polymer such as i) a polyoxyethylene-polyoxypropylene co-polymer or block co-polymer, and ii) a cross-linked acrylic acid polymer, e.g. as hereinabove described, adapted for topical once-a-day administration to the eye.

Preferably both polymer/thickener components component i) and ii) are present in a weight ratio of between about 1:200 and about 1:5 or between about 1:50 and about 1:20.

The compositions of the present invention may further comprise (2) a tonicity enhancing agent. Suitable tonicity enhancing agents are, e.g.

2.1 ionic compounds, such as alkali metal or alkaline earth metal halides, such as CaCl2, KBr, KCl, LiCl, NaI, NaBr or NaCl, or boric acid, and/or

2.2 non-ionic compounds such as urea, glycerol, sorbitol, mannitol, propylene glycol, or dextrose.

Conveniently, sufficient tonicity enhancing agent is added to impart to the ready-for-use ophthalmic composition an osmolality between about 50 and about 1000 mOsmol, preferably between about 100 and about 400 mOsmol, more preferably between about 200 and about 400 mOsmol and even more preferably between about 280 and about 350 mOsmol.

For the adjustment of the pH, preferably to a physiological pH, addition of (3) a pharmaceutically acceptable buffer system to the compositions of the invention is contemplated. Examples of buffer substances are acetate, ascorbate, borate, hydrogen carbonate/carbonate, citrate, gluconate, lactate, phosphate, propionate and tromethamine (tris-(hydroxymethyl)-aminomethane, TRIS) buffers. Tromethamine buffer is preferred. The buffer substance added is typically of an amount to ensure and maintain a physiologically tolerable pH range. The pH range is generally in the range between about pH 4 and about pH 9, preferably between about pH 4.5 and about pH 8.5 and more preferably between about pH 5.0 and about 8.2.

The compositions of the present invention may further comprise (4) a preservative to inhibit microbial growth during storage or after opening a closed container holding such a composition and exposing such a composition to the air. Useful preservatives are, e.g.,

4.1 a quaternary ammonium compound such as e.g. benzalkonium chloride (N-benzyl-N-(C₈-C₁₈-alkyl)-N,N-dimethylammonium chloride), benzoxonium chloride, cetrimide (hexadecyltrimethylammonium bromide) or the like.

4.2 alkyl-mercury salts of thiosalicylic acid, such as e.g. thiomersal, phenylmercuric nitrate, phenylmercuric acetate or phenylmercuric borate,

4.3 parabens, such as e.g. methylparaben or propylparaben,

4.4 alcohols, such as e.g. chlorobutanol, benzyl alcohol or phenyl ethanol,

4.5 biguanide derivatives, such as e.g. chlorohexidine or polyhexamethylene biguanide,

4.6 sodium perborate,

4.7 imidazolidinyl urea as known and commercially available under the trade name Germal®II,

4.8 sorbic acid,

4.9 stabilized oxychloro complexes such as known and commercially available under the trade name Purite®,

4.10 polyglycol-polyamine condensation resins, such as known and commercially available e.g. under the trade name Polyquart® from Henkel KGaA, and/or

4.11 a mixture of any components 4.1 to 4.10.

Preferred preservatives are quaternary ammonium compounds, in particular benzalkonium chloride and cetrimide. Where appropriate, a sufficient amount of preservative is added to the ophthalmic composition to ensure protection against secondary contamination during use by bacteria and fungi. The preferred preservatives are generally present in an amount between about 0.001% and about 0.02%.

The pharmaceutical compositions of the invention may additionally comprise (5) a solubilizer. Solubilizers suitable for the compositions of the invention are, e.g.:

5.1 octylphenoxy-poly(ethylenoxy)-ethanol (tyloxapol) known and commercially available under the trade name Triton®, e.g. Triton ® WR 1339, (Fiedler, loc. cit., p 1609),

5.2 polyethylene glycol glyceryl fatty acid ester. The fatty acid ester may include mono and/or di and/or tri fatty acid ester. The fatty acid constituent may include both saturated and unsaturated fatty acids having a chain length of from e.g. C₈-C₂₀. The polyethylene glycols may have e.g. from 5 to 40 [CH₂—CH₂—O] units, e.g. 5 or 30 units. Particularly suitable is polyethylene glycol (15) glyceryl monostearate or polyethylene glycol (15) glyceryl monooleate which is commercially available, e.g. under the trade name TGMS®-15 or TGMO®-15, respectively, e.g. from Nikko Chemicals Co., Ltd. Further suitable is polyethylene glycol (30) glyceryl monooleate which is commercially available, e.g. under the trade name Tagat® O, e.g. from Goldschmidt (H. Fiedler, loc cit, vol. 2, p. 1502-1503). Further suitable are polyethylene glycol glyceryl C₈-C₁₀ fatty acid ester with from 5 to 10 [CH₂—CH₂—O] units, e.g. 7 units, e.g. Cetiol® HE, or Labrasol®

5.3 polyoxyethylene C₈₋₂₀ fatty acid esters, e.g. polyoxyethylene stearic acid esters of the type known and commercially available under the trade name Myrj® (Fiedler, loc. cit., 2, p. 1042) or Brij® (Fiedler, loc. cit., p. 259; Handbook of Pharmaceutical Excipients, loc. cit., p. 367). An especially preferred product of this class is Myrj® 52 having a D²⁵ of about 1.1, a melting point of about 40 to 44° C., an HLB value of about 16.9, an acid value of about 0 to 1 and a saponification value of about 25 to 35,

5.4 glycerol ethers (Fiedler, loc. cit., p.701),

5.5 cyclodextrins, e.g. α-, β- or γ-cyclodextrin, e.g. alkylated, hydroxyalkylated, carboxyalkylated or alkyloxycarbonyl-alkylated derivatives, or mono- or diglycosyl-□-, □- or □-cyclodextrin, mono- or dimaltosyl- α-, β- or γ-cyclodextrin or panosyl-cyclodextrin, e.g. such as known and commercially available under the trade name Cavamax® or Cavasol® from Wacker Chemie. An especially preferred product of this class is hydroxypropyl-β-cyclodextrin, e.g. as known and commercially available under the trade name Cavasol® W7 HP or Cavasol® W8 HP. A mixture of cyclodextrins may also be used.

5.6 polyoxyethylene-sorbitan- C₈₋₂₀ fatty acid esters (polysorbates) e.g. produced by co-polymerising ethylene oxide with fatty acid esters of a sorbitol and its anhydrides of e.g. mono- and tri-lauryl, palmityl, stearyl and oleyl esters e.g. of the type known and commercially available under the trade name Tween® (Fiedler, loc.cit., p.1615) including the products Tween®

20 [polyoxyethylene(20)sorbitanmonolaurate],

21 [polyoxyethylene(4)sorbitanmonolaurate],

40 [polyoxyethylene(20)sorbitanmonopalmitate],

60 [polyoxyethylene(20)sorbitanmonostearate],

65 [polyoxyethylene(20)sorbitantristearate],

80 [polyoxyethylene(20)sorbitanmonooleate],

81 [polyoxyethylene(5)sorbitanmonooleate],

85 [polyoxyethylene(20)sorbitantrioleate].

Especially preferred products of this class are Tween®20 and Tween®80.

5.7 reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g. by reaction of a natural or hydrogenated castor oil or fractions thereof with ethylene oxide, e.g. in a molar ratio of from about 1:35 to about 1:60, with optional removal of free polyethylene glycol components from the product, e.g. in accordance with the methods disclosed in German Auslegeschriften 1,182,388 and 1,518,819. Especially suitable are the various tensides available under the trade name Cremophor. Particularly suitable are the products Cremophor RH 40 having a saponification no. ca. 50-60, an acid no.=<1, an iodine no.=<1, a water content (Fischer)=<2%, an n_{D}⁶⁰=ca.1,453-1,457 and an HLB=ca. 14-16; Cremophor RH 60 having a saponification no.=ca. 40-50, an acid no.=<1, an iodine no.=<1, a water content (Fischer)=ca. 4.5-5.5%, an n_{D}²⁵=ca.1.453-1,457 and an HLB=ca.15-17; and Cremophor EL having a molecular weight (by steam osmometry)=ca. 1630, a saponification no.=ca. 65-70, an acid no.=ca. 2, an iodine no.=ca. 28-32 and an n_{D}²⁵=ca.1.471 (c.f. Fiedler loc. cit. p. 326-327). Also suitable for use in this category are the various tensides available under the trade name Nikkol, e.g. Nikkol HCO-60. The said product NIKKOL HCO-60 is a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0° C.; Freezing point=ca. 32.4° C.; H₂O content (%, KF)=ca. 0.03, and/or

5.8 mixtures of the components 5.1 to 5.7.

Especially preferred solubilizers are Cremophor EL, Cremophor RH 40, tyloxapol and cyclodextrins. The concentration used depends especially on the concentration of the active ingredient. The amount added is typically sufficient to solubilize the active ingredient. For example, the concentration of the solubilizer can be between about 0.1 and about 5000 times the concentration of the active ingredient, preferably between about 0.5 and about 1000 times, more preferably between about 1 and about 500 times.

The compositions of the present invention may comprise additional excipients that can, e.g., function as a combined stabilizer/solubilizer. Such a combined additional stabilizer/solubilizer is for example a cyclodextrin or a mixture of cyclodextrins. A preferred cyclodextrin is in particular selected from the group of α-cyclodextrin, β-cyclodextrin, γ-cyclodextrin, hydroxypropyl-α-cyclodextrin, hydroxypropyl-β-cyclodextrin, dimethyl-α-cyclodextrin, randomly methylated β-cyclodextrin and dimethyl-β-cyclodextrin. The amount is generally in the range between about 0.01 and about 90% by weight, more preferably between about 0.1 and about 20% by weight.

The ophthalmic compositions may comprise further pharmaceutically acceptable excipients, such as (6) emulsifiers, (7) wetting agents or (8) fillers, such as, e.g. the polyethylene glycols (Fiedler, loc. cit., p. 2108, Handbook of Pharmaceutical Excipients, loc. cit., p 392) such as PEG 200, 300, 400 and 600, or Carbowax® 1000, 1500, 4000, 6000 and 10000.

Other exciplents that may be used if desired are listed below but they are not intended to limit in any way the scope of the possible excipients. They are especially (9) complexing agents, such as disodium-ethylenediamine tetraacetate, ethylenediamine tetraacetic acid (EDTA) in concentrations between, e.g., 0.005% and 0.05%, such as about 0.01%, and those complexing agents identified as physiologically compatible salts of phosphonic acids in U.S. Pat. No. 5,607,698 (10) antioxidants, such as ascorbic acid, acetylcysteine, cysteine, sodium hydrogen sulfite, butylated hydroxyanisole, butylated hydroxytoluene or alpha-tocopherol acetate; (11) stabilizers, such thiourea, thiosorbitol, sodium dioctyl sulfosuccinate or monothioglycerol; or (12) other excipients, such as, for example, lauric acid sorbitol ester, triethanol amine oleate or palmitic acid ester. Preferred exipients are complexing agents, such as disodium-EDTA. The amount and type of excipient added is in accordance with the particular requirements and is generally present in an amount between about 0.0001 and about 90% by weight.

In another embodiment, the present invention provides for compositions further comprising (13) an ophthalmic carrier. Such carriers are typically adapted for topical administration, and are for example

13.1 water,

13.2 mixtures of water and water-miscible solvents, such as C₁- to C₇-alkanols,

13.3 vegetable oils or mineral oils comprising from 0.5 to 5% by weight hydroxyethylcellulose, ethyl oleate, carboxymethyl-cellulose, polyvinyl-pyrrolidone,

13.4 water-soluble polymers for ophthalmic uses, such as, for example, cellulose derivatives, such as methylcellulose, alkali metal salts of carboxy-methylcellulose, hydroxymethylcellulose, hydroxyethylcellulose, methylhydroxypropyl-cellulose and hydroxypropylcellulose,

13.5 acrylates or methacrylates, such as salts of polyacrylic acid or ethyl acrylate, polyacrylamides,

13.6 natural products, such as gelatin, alginates, pectins, tragacanth, karaya gum, gellan gum such as Gelrite®, xanthan gum, carrageenin, agar and acacia,

13.7 starch derivatives, such as starch acetate and hydroxypropyl starch,

13.8 synthetic products, such as polyvinyl alcohol, polyvinylpyrrolidone, polyvinyl methyl ether, polyethylene oxide, or

13.9 mixtures of those polymers.

Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, or mixtures thereof The concentration of the carrier is typically between about 1 and about 10, 000 times the concentration of the active ingredient.

It will be appreciated that although the excipients have been described above by reference to a particular function any particular excipient may have alternative or multiple functions, e.g. cyclodextrin or a mixture of cyclodextrins may act as e.g. stabilizer, complexing agent and/or solubilizer.

The compositions of the present invention are surprisingly stable, as indicated by conventional tests, e.g. under stressed conditions, such as 15 hours at 80° C. or 1 month at 40° C. The compositions of the present invention are stable over 2, even 3, years showing less than 5% degradation of the ascomycin at 20 to 30° C. This surprising stability is also observed in an aqueous composition comprising anascomycin. Such aqueous compositions represent the preferred ophthalmic compositions of the present invention and contain an ascomycin typically in suspension.

A typical example of an aqueous ascomycin suspension contains:

| 1% Suspension | % | mg/mL | |
|---|---|---|---|
| 33-epi-chloro-33-desoxy- | 1% | 10 | mg/mL |
| ascomycin. | | | |
| (Micronised) | | | |
| Pluronic F127 NF (Poloxamer | 0.1% | 1.0 | mg/mL |
| 407) | | | |
| Carbopol 934P NF | 0.2% | 2.0 | mg/mL |
| Mannitol, USP | 4.3% | 43 | mg/mL |
| Benzalkonium Chloride | 0.015% | 0.15 | mg/mL |

| | | |
|---|---|---|
| Sodium Hydroxide | *Adjust to pH 6.5 | *Adjust to pH 6.5 |
| Water for injection | qs 400 g | qs 400 g |

The present invention is therefore also useful to stabilize an aqueous composition comprising an ascomycin, in particular comprising an ascomycin suspension.

The ophthalmic compositions of the present invention may be prepared in conventional manner e.g. by mixing an ascomycin and appropriate excipients.

Filling may be effected before or after sterilization of the resulting mixture. Sterilization of the composition of the present invention and the primary package can be effected e.g. by gamma irradiation, by ethylene oxide treatment, by electron beam, by autoclaving, by microwave treatment, by filtration through a sterile filter, or by steam sterilization.

The compositions of the present invention may be packaged in conventional manner. The compositions of the present invention may be stored in single or multiple unit dosage form, e.g. closed bottles, tubes or other containers made from glass, plastic such as e.g. polyethylene, polyethylene terephthalate, or polypropylene, or metal or combinations thereof. For example bottles may contain about 1 to 10 ml of the compositions of the present invention. The container may be fitted with a dropper to facilitate administration.

The compositions of the present invention may be formulated in conventional manner e.g. to be particularly adapted for topical ophthalmic use. In so far as the procedures for formulation are not particularly described herein such formulation procedures may for example be known in the art, or analogous to those known in the art or to procedures described herein. Representative procedures are disclosed in for example, Remington's Pharmaceutical Sciences, 19th Ed., Mack Publ., Co., 1995, H. Sucker et al, Pharmazeutische Technologie, 2nd Edition, Thieme, 1991, R: H. Mueller et al, Pharmazeutische Technologie: Moderne Arzneimittelformen, 2nd Edition, Wissenschaftliche Verlags-gesellschaft, Stuttgart, 1998, L. Lachman et al. The Theory and Practice of Industrial Pharmacy, 3rd Ed, 1986, and Hagers Handbuch der pharmazeutischen Praxis, 4th Ed. Vol. 7, (Springer Verlag, 1971) as well as later editions, the contents of all of which are incorporated herein by reference.

The excipients used may e.g. be those known in the art e.g in the Lexikon der Hilfsstoffe für Pharmazie, Kosmetik und angrenzende Gebiete; and Handbook of Pharmaceutical Excipients, references referred to above, or analogous to those known in the art or new excipients having analogous function to those described in the art or herein.

The compositions of the present invention are useful for the treatment of immune-mediated conditions of the eye, such as auto-immune diseases, e.g. dry eye, uveitis, keratoplasty or chronic keratitis; allergic conditions, e.g. vernal conjunctivitis; inflammatory conditions or corneal transplants, and in particular of dry eye, and may be used for the treatment and prevention of signs and symptoms of the ocular conditions as indicated e.g. in standard animal trials and clinical trials.

One animal test comprises a modified Draize test on three albino rabbits wherein the ocular tolerability after a single dose instillation of 50 microlitres of compositions of the present invention on the ocular surface is shown for the 15 minutes after instillation then after 1, 2 and 7 days. The tolerability is based on visual examination considering the following parameters: discomfort as judged by blinking or partial/complete closure of the eye, duration of discomfort, discharge, redness of conjunctiva (palpebral and bulbar conjunctiva), chemosis of conjunctiva (swelling), degree of opacity of cornea and area of cornea involved, and pathological influence upon iris.

A clinical trial may be effected to test the efficacy and tolerability of about 30 to 40 microliters of compositions of the present invention containing 1% of an ascomycin administered once a day by instillation onto the ocular surface, e.g. to the inside lower lid, to groups of, e.g. 10 to 25, healthy volunteers, or patients suffering from allergic conjunctivitis. The trial lasts e.g. 8 days.

The subjects are examined to determine the effect against conjunctivitis, e.g. fast onset of action and long duration of action and good tolerability, e.g. lack of significant irritation or reddening.

Additionally the bioavailability of the compositions of the present invention in the above trials may be determined by absorption in the conjunctiva or surrounding tissues.

The bioavailability of an addressed once-a-day ophthalmic composition may be assessed with the pharmacokinetic assay described infra:

A fixed volume, e.g. 50 microliters, of the ophthalmic formulation is instilled onto the upper part of the conjunctiva of rabbits. Tears, bulbar conjunctiva, cornea and sclera are sampled after either 5, 15, or 30 minutes, or 1, 8, 16, or 20 hours. Samples are extracted for ascomycin determination related to the wet weight amount of tissue or tears. Ascomycin is determined using a liquid chromatography linked to mass spectrography (LC-MS) validated method.

The retention of an addressed once-a-day ophthalmic composition may be assessed with retention tests described infra:

The upper eyelid of one eye of an albino rabbit is carefully pulled away from the eyeball and 50 microliter of the test substance is instilled on the superior part of the bulbar conjunctiva using a gauged automatic pipette. The eyelid is gently closed for about one second. After either 5 min, 1 hour, 4 hours, 8 hours, 16 hours or 24 hours a weighed Schirmer's test strip is maintained in the cul-de-sac between the inferior lid and the temporal part of the eyeball for exactly one minute. The absorbed tears collected on each Schirmer test strip are immediately weighed and the ascomycin content is determined e.g. after extracting the strips by liquid chromatography/negative ion chemical ionization mass spectrometry using a deuterated analogue of the ascomycin as internal standard. The strips may be kept frozen up to analytical test of the ascomycin content.

The exact amount of the ascomycin to be administered will naturally depend on a variety of factors, e.g. choice of salt, excipients, formulation properties, and severity of the condition. Conveniently, the composition of the present invention is administered to the cornea once a day, e.g. after breakfast. Preferably from about 25 to about 75 microlitres, e.g. from about 50 to about 75 microlitres, is administered, e.g. using a dropper.

The daily dose of the ascomycin to be administered is from about 1 micrograms/kg to about 5 micrograms/kg. For larger mammals, e.g. a 70 kg mammal such as a human, a dose of from about 100 to about 300 micrograms, is indicated.

Therefore, in a further aspect the present invention provides

a) an ophthalmic composition as defined above for use in the treatment of immune-mediated conditions of the eye, such as auto-immune diseases, e.g. dry eye, uveitis, keratoplasty or chronic keratitis; allergic conditions, e.g. vernal conjunctivitis; inflammatory conditions or corneal transplants, in particular dry eye, or a condition treatable by ascomycin therapy,

b) a method for treating immune-mediated conditions of the eye, such as auto-immune diseases, e.g. dry eye, uveitis, keratoplasty or chronic keratitis; allergic conditions, e.g. vernal conjunctivitis; inflammatory conditions or corneal transplants, in particular dry eye, or a condition treatable by ascomycin therapy comprising administering a composition of the present invention to the eye of a patient in need thereof, or

c) the use of a composition of the present invention in the preparation of a medicament for the treatment of immune-mediated conditions of the eye, such as auto-immune diseases, e.g. dry eye, uveitis, keratoplasty or chronic keratitis; allergic conditions, e.g. vernal conjunctivitis; inflammatory conditions or corneal transplants, in particular dry eye, or a condition treatable by ascomycin therapy. All percentages referred to herein are weight/weight except where otherwise indicated.

1. An ophthalmic composition comprising an ascomycin and at least one polymer selected from the group consisting of
i) a polyoxyethylene-polyoxypropylene co-polymer, and
ii) a cross-linked acrylic acid polymer.

2. A composition according to claim 1 comprising both a polyoxyethylene-polyoxypropylene block co-polymer, and a cross-linked acrylic acid polymer that is cross-linked with a polyol.

3. A composition according to claim 2 further comprising water, a tonicity enhancing agent, a buffer, and a preservative.

4. A composition according to claim 1 wherein the ascomycin is 33-epi-chloro-33-desoxy-ascomycin.

5. A composition according to claim 4 wherein said a polyoxyethylene-polyoxypropylene co-polymer is a polyoxyethylene-polyoxypropylene block co-polymer and said cross-linked acrylic acid polymer is cross-linked with a polyol.

6. A composition according to claim 5 wherein said polyol is a member selected from the group consisting of allylsucrose or allylpentaerythritol.

7. A method for treating immune-mediated conditions of the eye, allergic conditions, inflammatory conditions, or corneal transplants comprising administering an effective amount of a composition according to claim 1 to the eye of a patient in need thereof.

8. A method for treating dry eye comprising administering an effective amount of a composition of claim 1 to the eye of a patient in need thereof.

9. The method of claim 7 wherein the condition is selected from the group consisting of auto-immune diseases, dry eye, uveitis, keratoplasty, chronic keratitis and vernal conjunctivitis.

10. The method of claim 7 wherein the ascomycin is 33-epi-chloro-33-desoxy-ascomycin.

11. The method of claim 8 wherein the ascomycin is 33-epi-chloro-33-desoxy-ascomycin.

12. The method of claim 9 wherein the ascomycin is 33-epi-chloro-33-desoxy-ascomycin.

13. A method for treating immune-mediated conditions of the eye, allergic conditions, inflammatory conditions, or corneal transplants comprising administering an effective amount of a composition according to claim 2 to the eye of a patient in need thereof.

14. A method for treating dry eye comprising administering an effective amount of a composition of claim 2 to the eye of a patient in need thereof.

15. The method of claim 13 wherein the condition is selected from the group consisting of auto-immune diseases, dry eye, uveitis, keratoplasty, chronic keratitis and vernal conjunctivitis.

16. The method of claim 13 wherein the ascomycin is 33-epi-chloro-33-desoxy-ascomycin.

17. The method of claim 14 wherein the ascomycin is 33-epi-chloro-33-desoxy-ascomycin.

18. The method of claim 15 wherein the ascomycin is 33-epi-chloro-33-desoxy-ascomycin.
